(19) United States
(12) Patent Application Publication
(10) Pub. No.: US 2008/0255482 A1
(43) Pub. Date: Oct. 16, 2008

(54) INTRATHORACIC PRESSURE LIMITER AND CPR DEVICE FOR REDUCING INTRACRANIAL PRESSURE AND METHODS OF USE

(75) Inventor: Keith G. Lurie, Minneapolis, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Advanced Circulatory Systems, Inc., Eden Prairie, MN (US)

(21) Appl. No.: 11/735,924

(22) Filed: Apr. 16, 2007

(51) Int. Cl. A61H 31/00; A61N 1/00

(52) U.S. Cl. 601/43; 601/41; 607/4

(57) ABSTRACT

In certain aspects, the present invention relates to CPR devices that are designed to limit the rise in intrathoracic pressure when performing CPR compressions (e.g., chest, thorax, etc.) so as to minimize increases in intracranial pressure resulting from such compressions and elevated ITP. Exemplary CPR devices of the invention generally include one or more pressure sensors and a control system configured to adjust or alert for the need to adjust the amplitude and duration of CPR compressions so as to prevent a rise in ITP above a certain threshold amount, thereby controlling the rise in ICP during CPR. Methods of controlling a rise in ICP during CPR are also provided.

### BACKGROUND OF THE INVENTION

The present invention relates generally to devices and methods used in conjunction with cardiopulmonary resuscitation procedures.

Worldwide, sudden cardiac arrest is a major cause of death and is the result of a variety of circumstances, including heart disease and significant trauma. In the event of a cardiac arrest, several measures have been deemed to be essential in order to improve a patient's chance of survival. These measures must be taken as soon as possible to at least partially restore the patient's respiration and blood circulation. Severe hypotension or very low blood pressure can lead to passing out and in some circumstances cardiac arrest. Like cardiac arrest, patients with low blood pressure often suffer from insufficient blood returning to the heart after each beat. This results in a decrease in forward blood flow out of the heart and eventually to low blood pressure.

One common technique, developed more than 40 years ago, is an external chest compression technique generally referred to as cardiopulmonary resuscitation (CPR). CPR techniques have remained largely unchanged over the past decades.

With traditional CPR, pressure is applied to a patient's chest in order to increase intrathoracic pressure. An increase in intrathoracic pressure induces blood movement from the region of the heart and lungs towards the peripheral arteries. Such pressure partially restores the patient's circulation. Traditional CPR is performed by actively compressing the chest by direct application of an external pressure to the chest. After active compression, the chest is allowed to expand by its natural elasticity which causes expansion of the patient's chest wall. This expansion allows some blood to enter the cardiac chambers of the heart. The procedure as described, however, is insufficient to ventilate the patient. Consequently, conventional CPR also requires periodic ventilation of the patient. This is commonly accomplished by mouth-to-mouth technique or by using positive-pressure devices, such as a self-inflating bag which relies on squeezing an elastic bag to deliver air via a mask, endotracheal tube or other artificial airway.

In order to increase cardiopulmonary circulation induced by chest compression, a technique referred to as active compression-decompression (ACD) has been developed. According to ACD techniques, the active compression phase of traditional CPR is enhanced by pressing an applicator body against the patient's chest to compress the chest. Such an applicator body is able to distribute and apply force substantially evenly over a portion of the patient's chest. More importantly, however, the applicator body is sealed against the patient's chest so that it may be lifted to actively expand the patient's chest during the decompression step. The resultant negative intrathoracic pressure induces venous blood to flow into the heart and lungs from the peripheral venous vasculature of the patient. The present inventors have also developed intrathoracic pressure regulator (ITPR) and inspiratory impedance threshold devices (ITD) to aid in generation of negative intrathoracic pressures.

Also of importance to the invention are ventilation sources that are used in connection with CPR techniques to properly ventilate the patient. One type of ventilation source is the AMBU bag available from AMBU International, Copenhagen, Denmark. The AMBU bag can also be used in connection with a positive end-expiratory pressure (PEEP) valve, available from AMBU International, to treat some patients with pulmonary and cardiac diseases.

ACD-CPR techniques are described in detail in Todd J. Cohen et al., Active Compression-Decompression Resuscitation: A Novel Method of Cardiopulmonary Resuscitation, American Heart Journal, Vol. 124, No. 5, pp. 1145-1150, November 1992; and Todd J. Cohen et al., Active Compression-Decompression: A New Method of Cardiopulmonary Resuscitation, The Journal of the American Medical Association, Vol. 267, No. 21, Jun. 3, 1992. These references are hereby incorporated by reference.

The use of a vacuum-type cup for actively compressing and decompressing a patient's chest during ACD-CPR is described in a brochure of AMBU International A/S, Copenhagen, Denmark, entitled Directions for Use of AMBU® CardioPump™, published in September 1992. The AMBU® CardioPump™ is also disclosed in European Patent Application No. 509 773 A1. These references are hereby incorporated by reference.

Despite these methodologies, there exists a need for improvements.

### BRIEF SUMMARY OF THE INVENTION

To address such needs and others, in certain aspects of the invention, a device for controlling rise in intracranial pressure (ICP) during cardiopulmonary resuscitation (CPR). In accordance with certain embodiments, the device includes: an intrathoracic pressure (ITP) sensor; and a control system configured to adjust or alert for the need to adjust the amplitude and duration of CPR compressions to prevent a rise in ITP above a certain amount. This amount could be about 25 mmHg, at the time of maximum chest compression, and in other cases about 50 mmHg, although other thresholds are possible. These values can be assessed by measuring airway pressures, for example, which can serve as a surrogate for intrathoracic pressure. This can be achieved by using a pressure transducer in the airway and by transiently occluding the airway (e.g. endotracheal tube) during the chest compression phase of CPR. Since changes in intrathoracic pressure are directly related to changes in intracranial pressure, and excessively high levels of intracranial pressure can cause brain damage, measurement of airway pressures and subsequent adjustment of the compression force can be used as a way to reduce the potential for excessively high and harmful elevation in intracranial pressures. In this way, a process is provided for controlling the rise in ICP during CPR. The devices of the invention may also include a mechanism for increasing cardiopulmonary circulation through CPR compressions, wherein the mechanism is configured to allow for decompression after each compression.

In other aspects of the invention, a device for controlling rise in intracranial pressure (ICP) during cardiopulmonary resuscitation (CPR) is provided, wherein the device includes a mechanism for increasing cardiopulmonary circulation through CPR compressions, wherein the mechanism is configured to allow for decompression after each compression; a direct or indirect intracranial pressure (ICP) sensor; and a control system configured to adjust or alert to adjust the amplitude and duration of CPR compressions to prevent a rise in peak ICP above about 25 mmHg and in some cases above about 50 mmHg.

In yet other aspects of the invention, a method for controlling the rise in intracranial pressure (ICP) during cardiopulmonary resuscitation (CPR) is provided. The method generally includes: performing CPR to a subject in need thereof; directly or indirectly assessing ICP of the subject; and adjusting the degree of CPR compressions if the subject's peak ICP is determined to be above about 25 mmHg and in some cases above about 50 nm mHg. In accordance with certain embodiments, the subject's ICP may be assessed via a device comprising an ICP sensor and/or an intrathoracic pressure (ITP) sensor; and a control system configured to adjust or alert to the need to adjust the degree of CPR compressions if the subject's ICP is determined to be above a certain threshold amount. In some cases, this could be about 25 mmHg and in some cases above about 50 mmHg.

In one embodiment, the CPR may be performed via an automated compression device, and the control system is configured to automatically adjust the amplitude and duration of CPR compressions to prevent a rise in ICP above a certain amount, which could be above about 25 mmHg, or above about 50 mmHg, based at least in part on readings from the ICP and/or ITP sensor. In another embodiment, the CPR may be performed manually by a CPR administrator, and the control system visually and/or audibly alerts the CPR administrator to adjust the amplitude and duration of CPR compressions so as to prevent a rise in ICP above a certain amount, such as above about 25 mmHg or above about 50 mmHg, based at least in part on readings from the ICP and/or ITP sensor.

These and other aspects of the invention will be described in more detail throughout the present specification and more particularly below in conjunction with the following drawings.

### BRIEF DESCRIPTION OF THE DRAWINGS

FIG. 1 illustrates an embodiments of a CPR device in accordance with certain aspects of the invention.

FIG. 2 illustrates an exemplary CPR methodology in accordance with certain aspects of the invention.

FIG. 3 illustrates real time tracings of ICP (intracranial pressure) and ETP (endotracheal pressure) during spontaneous breathing and baseline without a device and during breathing through an impedance threshold device with an inspiratory resistance of −10 and −15 mmHg.

FIG. 4 illustrates real time tracings of ICP (intracranial pressure) and ETP (endotracheal pressure) after successful resuscitation from untreated ventricular fibrillation, during baseline spontaneous breathing and during breathing through an ITD with −10 and −15 mmHg of ETP, including elevated baseline ICP due to preceding cardiac arrest.

FIG. 5 illustrates a total of 20 minutes of real time tracing showing endotracheal pressure (ETP), aortic pressure (AoP), intracranial pressure (ICP) and right atrial pressure (RAP) of a non-breathing animal after a 35% blood loss.

FIG. 6 illustrates that following controlled 40% bleed in a pig model of shock, an ITPR set at −15 mmHg significantly improves aortic pressure and decreases intracranial pressure.

### DETAILED DESCRIPTION OF THE INVENTION

A further understanding of the nature and advantages of the present invention may be realized by reference to the remaining portions of the specification and the drawings wherein like reference numerals are used throughout the several drawings to refer to similar components. Further, all references, patents and publications cited herein are specifically incorporated by reference in their entirety for all purposes.

The present invention generally relates to devices and methods for use in cardiopulmonary resuscitation (CPR), which are designed to control the rise in intracranial pressure during CPR. The popular teaching is to push hard and push fast. However, pushing too hard can have detrimental effects, especially if you restrict the outward movement of the ribs when the sternum is compressed, as the ribs serve as a pressure relief system to help reduce the amount of ITP. Without some pressure relief, CPR methodologies can increase the ITP too high: while this increases arterial pressure it also increases ICP, thereby reducing blood flow to the brain.

While the effects of positive intrathoracic pressure (ITP) on intracranial pressure (ICP) have been known, the impact of subatmospheric intrathoracic pressure changes on pressures in the brain is less well characterized. In the course of studying an inspiratory impedance threshold device (ITD) to reduce intrathoracic pressure during the decompression phase of cardiopulmonary resuscitation, it was recently observed by the certain of the present inventors that subatmospheric pressures generated during the chest wall recoil phase of CPR caused a decrease in intracranial pressure (ICP). In these CPR studies, positive pressure ventilation resulted in an immediate increase in ICP, whereas each time the chest wall recoiled and a negative pressure was generated in the thorax the ICP decreased. In addition, it has recently been demonstrated that in pigs in cardiac arrest, gasping during untreated ventricular fibrillation decreases immediately intracranial pressure and increases cerebral perfusion pressure.

Based upon this observed relationship between intrathoracic pressure changes and intracranial pressure, mechanical devices designed to reduce intrathoracic pressure have been tested in spontaneously breathing as well as apneic euvolemic and hypovolemic hypotensive pigs to investigate effects of ITP on ICP, in a dose related manner. Thus, in certain aspects of the invention, it has been found that ICP can be regulated by control of ITP. Investigations have found effects both in spontaneously breathing animals with the inspiratory impedance threshold device (ITD) and in apneic animals with the intrathoracic pressure regulator device (ITPR). In other aspects of the invention, this regulation of ICP is applied in cardiopulmonary resuscitation (CPR) techniques to prevent a rise in ICP during CPR compressions.

Without intending to be limited by theory, in certain aspects of the invention, it has been found that the rise in ICP is directly related to the degree of CPR compression, and that the effects of the repetitive physical pounding of such compressions during CPR on the brain is a likely cause of the high incidence of brain damage and mortality rates associated with CPR. In accordance certain aspects of the invention, it has been found that both increases and decreases in ITP result in nearly instantaneous increases and decreases in intracranial pressure.

While some rise in ITP is critical to force blood out of the heart to the rest of the body with each CPR compression, if the repetitive CPR compressions cause too high a rise in ITP, that pressure is directly transmitted to the brain primary via the spinal cord and ICP rises simultaneously with the increase in arterial pressure to the brain. The brain is squeezed, and there is no blood perfusion of the brain (cerebral perfusion pressures can be calculated as the difference between the mean arterial pressure and the mean ICP). The increase in ICP creates a “resistance” in the fluid-filled circuitry that includes the arteries to the brain and the venous drainage out of the brain. The higher the ICP, the less blood that actually gets to the brain tissue.

### I. CPR Devices

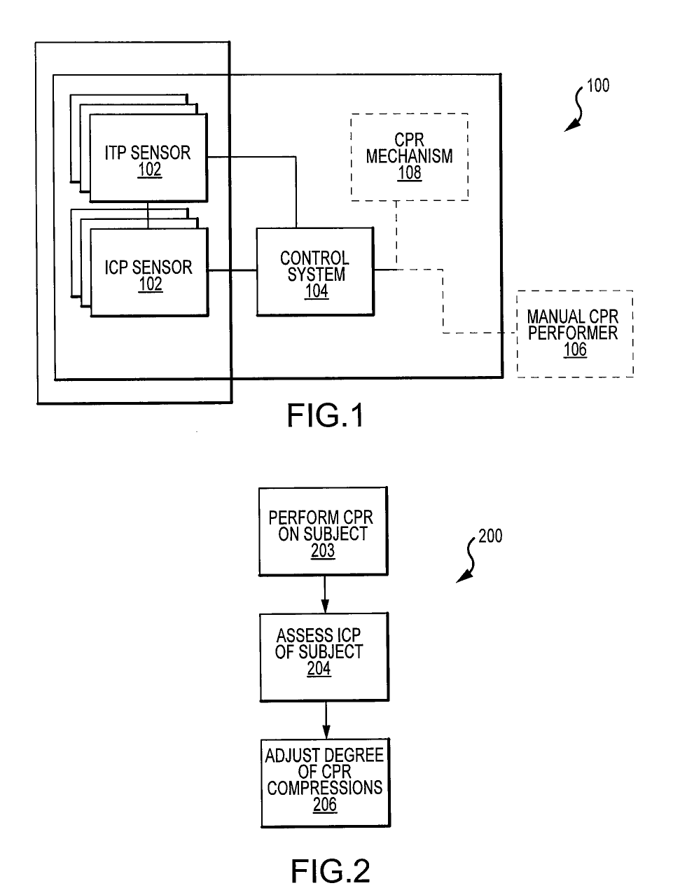

In certain aspects, the present invention relates to CPR devices that are designed to limit the rise in ITP when performing CPR compressions (e.g., chest, thorax, stomach, etc.) so as to minimize increases in ICP resulting from such compressions and elevated ITP. With reference to FIG. 1 (optional components shown in dashed lines), exemplary CPR devices 100 of the invention generally include one or more pressure sensors 102 and a control system 104 configured to adjust or alert for the need to adjust the amplitude and duration of CPR compressions so as to prevent a rise in ITP above about 50 mmHg, 40 mmHg, and 25 mmHg, etc., thereby controlling the rise in ICP during CPR. In certain embodiments, the rise in peak ICP is controlled so as to not rise above about 50 mmHg, 40 mmHg, and 25 mmHg, etc., as recognized by those skilled in the art, so as to prevent damage to the brain and its tissues. It is recognized that non-invasive means to measure cerebral perfusion and perfusion pressure can serve as a surrogate for the direct measurement of ICP.

By way of example, in certain embodiments, using pressure sensors 102 and a feedback control system 104, the CPR device 100 may alert a performer or administrator of CPR 106 to adjust the amplitude and duration of CPR compressions to thereby limit of the rise of ITP, to optimize brain perfusion without causing brain damage due to elevated ICP. In other embodiments, the CPR compressions may be performed automatically via mechanism for increasing cardiopulmonary circulation through CPR compressions 108, and the control system 104 may automatically adjust the amplitude and duration of CPR compressions.

In certain embodiments, the pressure sensor(s) 102 may monitor intrathoracic pressure and/or intracranial pressure, either directly or indirectly. In certain embodiments, the ITP and/or ICP sensors may be non-invasive physiological pressure sensors. For instance, the sensor may sense physiological parameters that change proportionally with ICP and/or ITP, including but not limited to: airway pressures, jugular venous pressures, and changes in impedance in the neck and spinal column. Increases and decreases in ITP, as measured directly or indirectly (for example with a sensor on an endotracheal tube) track in parallel with changes in ICP.

By way of non-limiting example, measurement of electrical impedance in the neck, with the electrical vector transecting the spinal cord, can provide direct and instantaneous measurement capability of the changes in ICP. ICP measured in the spinal cord, including in the cervical portion of the spinal cord, directly reflect changes in ICP measured in the brain. ICP measurement can also be accomplished by measuring the changes in pressure in the eye ball and optic nerve. ICP can also be measured indirectly based upon changes reflected in the neck by changes in jugular venous pressure. An increase in neck circumference at the level of the jugular veins and common carotid artery can also be used an indirect measure of ICP.

In one embodiment, the pressure sensor(s) may be located in the patient's airway, including in the nose, either attached to a breathing tube or other airway adjunct. In certain embodiments, the pressure sensor(s) may detect changes in intrathoracic pressure with each CPR compression and decompression. In other embodiments, the pressure sensor(s) may measure the changes in electrical impedance (i.e., bioimpedance), or the rate of change of electrical impedance (derivative of impedance) measured with, e.g., two or more electrodes, positioned to captures the changes in electrical impedance, e.g., in the cervical spinal column. Lower levels of impedance indicate that there is more spinal fluid in the cervical spine and thus higher ICPs.

In one exemplary embodiment, one or more pressure sensor(s) may be incorporated into an inspiratory impedance threshold device (ITD). As understood by those of skill in the art, an ITD prevents respiratory gases from entering the thorax during the decompression phase of CPR for some period of time. A modification of the ITD could be made that also periodically and transiently prevents respiratory gases from exiting the thorax, and thus enables the measurement of pressures in the airway for a very brief period of time with each compression, one compression out of 10, 100, etc. compressions, etc. By transiently occluding the airway for 10-2000 msec, the pressure within the thorax increases transiently and in direct proportion to the compression force. The measured value or change in slope of the measured values may then be used to direct an increase or decrease in the amount of external force applied with each compression. However, the occluding member does not remain in the closed (occluding) position for more than, e.g., 250 ms, and generally is not used with every compression.

In general, CPR device 100 may be regulated by ITP and/or ICP measurements (with optional control system 104 programming to instantaneously assess ICP) which provide feedback through a closed loop control system 104 to at least in part regulate CPR compressions, e.g., the forces applied to compress the sternum, compress the anterior chest wall, compress the thoracic in a circumferential manner (e.g., vest CPR) or compress the chest with a band-like device (e.g., Zoll Autopulse™).

In certain embodiments, the CPR device may include a mechanism for increasing cardiopulmonary circulation through CPR compressions 108. Generally, such mechanisms may be configured to allow for decompression as well. By way of example, the CPR mechanism may decompress the chest and/or compress and decompress the abdomen or other body parts (e.g., lower limbs) in a constant of repetitive manner in relation to the chest. The control system 104 generally interfaces with the CPR mechanism 108 to adjust the amplitude and duration of compressions (and optionally decompressions) to limit the rise in ITP, and the duration of the rise in ITP, so as to minimize the negative effects of the rise in ICP during CPR.

By way of example, the control system 104 may include computer executable code configured to adjust the amplitude and duration of CPR compressions (and/or optionally decompressions) when a peak ITP and/or ICP of greater than about 50 mmHg, about 40 mmHg, or about 25 mmHg etc. is detected. In other embodiments, the control system 104 may include computer executable code configured to alert for the need to adjust the amplitude and duration of CPR compressions (and/or optionally depressions). The alerts may be, e.g., visual (by way of a light, LED, or the like), or audible (by way of a horn, beep, or the like). An administrator of manual CPR or an operator of a CPR device may then adjust compressions and/or decompressions of CPR to control ITP and ICP to the desired pressure, e.g., below about 50 mmHg.

In accordance with certain aspects of the invention, it has been found that CPR devices thought to enhance blood pressure may be in fact very dangerous, as when the blood pressure is increased so is the ICP. In addition, it is important to take in account the potential phase shifts in the pressure waveforms as they are transmitted to the brain in large part through the para-vertebral sinuses (venous beds the interdigitate with the spinal column in the thoracic spine). The cerebral perfusion gradient is the difference between the arterial pressure and the afterload, usually calculated as the ICP. If the ITP and the ICP rise and fall out of phase, then perfusion is optimized. When the rise and fall together and to an equal degree, then there is no gradient. Phase shifts as a result of the devices of the invention may have a significant impact on brain perfusion. Thus, in certain aspects of the invention, control system 104 may optionally include computer executable code configured to optimize phase shift between ITP and ICP (based on readings from pressure sensors 102), to thereby optimize brain perfusion.

According to the present invention, the devices of the invention 100 may include any known mechanisms for increasing cardiopulmonary circulation induced by CPR compression (and optionally decompression) 108. For instance, such mechanisms include thoracic vest devices and thoracic strap devices, where a circumferential collar or strap is compressed in a repetitive manner to promote blood flow from the heart; CPR with a newly described Hyack Oscillator ventilatory system which operates essentially like an iron-lung-like device; phrenic nerve stimulators, including those described in U.S. application Ser. No. 09/095,916, filed Jun. 11, 1998; Ser. No. 09/197,286, filed Nov. 20, 1998; Ser. No. 09/315,396, filed May 20, 1999; and Ser. No. 09/533,880, filed Mar. 22, 2000, the complete disclosures of which are herein incorporated by reference; constant compression piston devices; interposed abdominal compression-decompression CPR devices; and active compression-decompression (ACD) CPR devices; and combination IAC-ACD CPR devices. In certain embodiments, the CPR mechanisms can include CPR with a thoracic vest, CPR with a thoracic strap (e.g., the Zoll AutoPulse™), active compression/decompression CPR, CPR performed with a piston (e.g., the Michigan Instrument's Thumper™), interposed abdominal counterpulsation (IAC) CPR, and CPR with a LifeStick™.

### II. CPR Methodologies

Other aspects of the invention relate to non-invasive CPR methodologies for controlling the rise in ICP during CPR. With reference to FIG. 2, the invention relates to non-invasive CPR method 200. Generally, in accordance with methods of the invention, at step 202, CPR is performed on a subject in need thereof, and ICP is assessed, either directly or indirectly, at step 204. Any known method may be used for performing CPR, as described above. By way of non-limiting example, CPR may be performed via manual CPR compression/decompression, CPR with a thoracic vest, CPR with a thoracic strap (e.g., the Zoll AutoPulse), active compression/decompression CPR, CPR performed with a piston (e.g., the Michigan Instrument's Thumper), interposed abdominal counterpulsation (IAC) CPR, and CPR with a LifeStick.

At step 204, ICP may be assessed via one or more pressure sensors 102, as described with reference to device 100. Thus, in certain embodiments, ICP may be assessed by ITP sensors and/or ICP pressure sensors. Moving on to step 206, the degree of CPR compressions (and optionally decompressions) may be adjusted if the subject's ICP is determined to be above about 50 mmHg, about 40 mmHg, about 25 mmHg, etc. Control system 104 may generally be used to adjust CPR compressions, or alert for the need to adjust CPR compression in step 206. In certain embodiments, the amplitude and duration of the CPR compressions (and/or optionally decompressions) may be adjusted to achieve the desired ICP below about 50 mmHg.

In accordance with the methods of the methods of the invention, the subject may be any subject which may benefit from administration of CPR techniques, as recognized by those skilled in the art. Further, the subject may be a mammal, including a human. In particular embodiments, the subject may be suffering from cardiac arrest, head trauma, low blood pressure and patients in right heart failure and in shock.

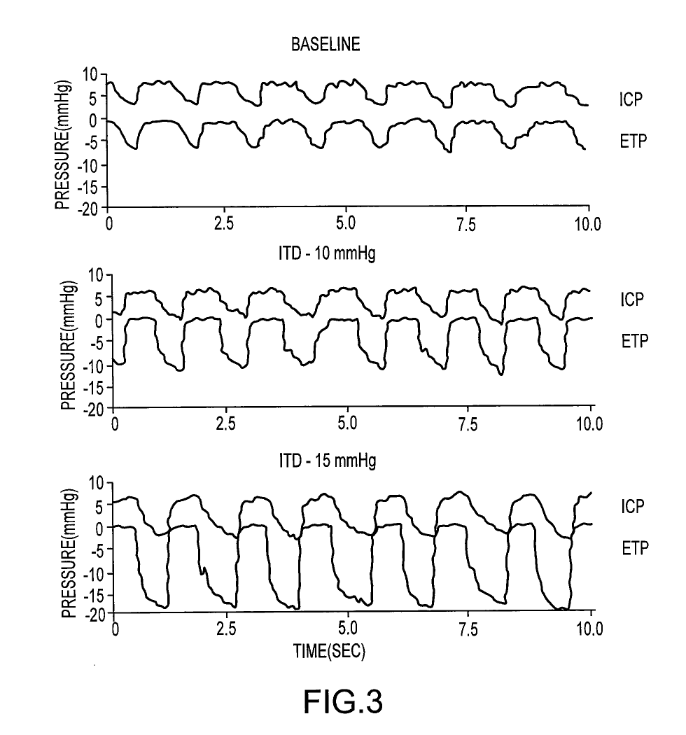

FIG. 3 illustrates one example of real time tracings of ICP (intracranial pressure) and ETP (endotracheal pressure) during spontaneous breathing and baseline without any type of airflow impedance device and during breathing through an impedance threshold device with an inspiratory resistance of −10 and −15 mmHg. As shown, each inspiration through the impedance device lowers endotracheal pressure (ETP) in the airway and simultaneously lowers intracranial pressure (ICP). The greater the inspiratory resistance, the lower the intrathoracic pressure and the lower the intracranial pressure. As such, FIG. 3 illustrates the relationship between airway pressure, thoracic pressure, and intracranial pressure.

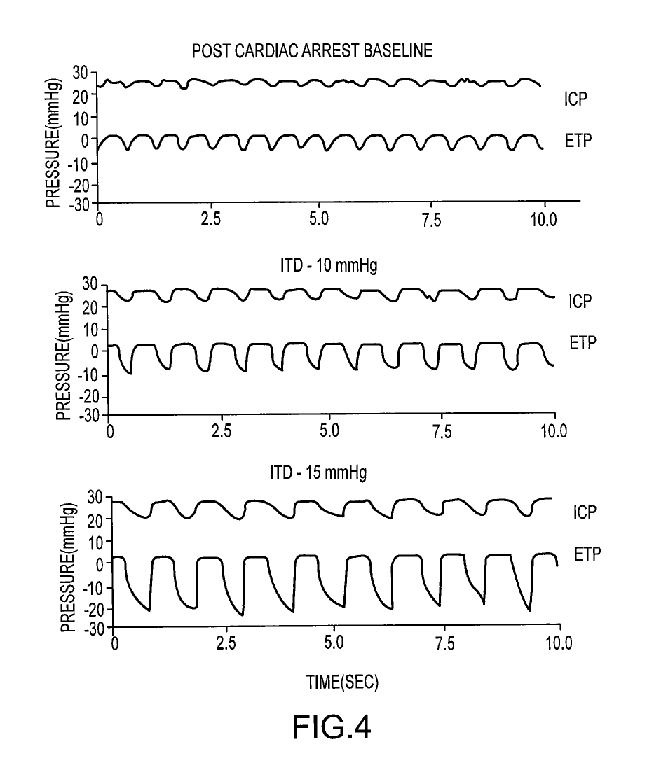

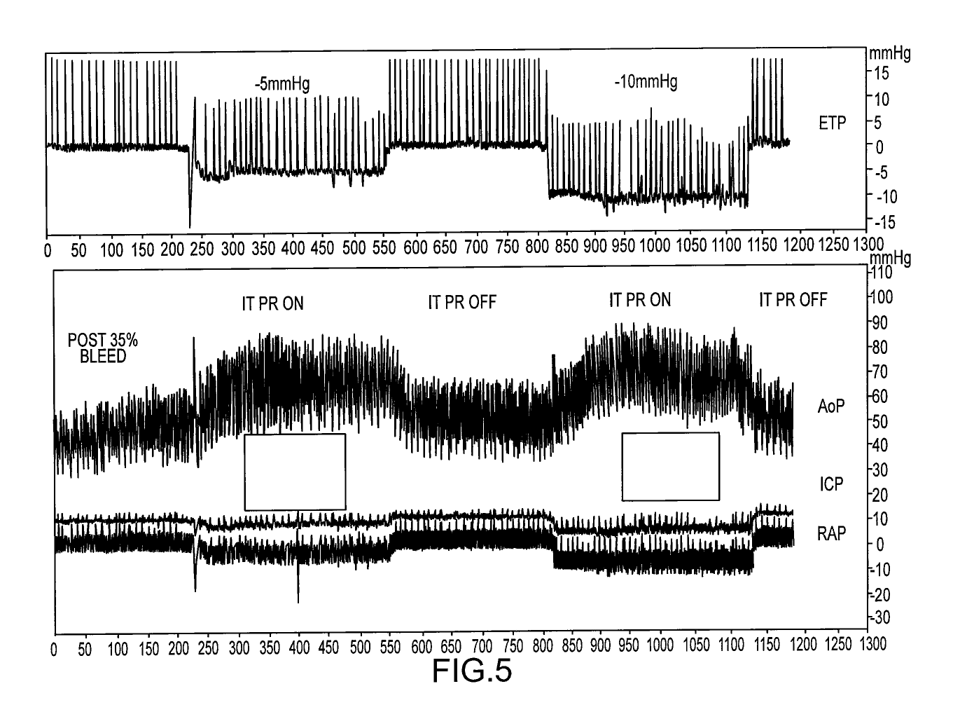

FIG. 4 illustrates real time tracings of ICP (intracranial pressure) and ETP (endotracheal pressure) after successful resuscitation from untreated ventricular fibrillation, during baseline spontaneous breathing and during breathing through an impedance threshold device ITD with a resistance or cracking pressure of −10 and −15 mmHg of ETP, including elevated baseline ICP due to preceding cardiac arrest. In this situation, the intracranial pressures are elevated after cardiac arrest and resuscitation. Breathing through the ITD lowers intrathoracic pressures and intracranial pressures.

FIG. 5 illustrates a total of 20 minutes of real time tracing showing endotracheal pressure (ETP), aortic pressure (AoP), intracranial pressure (ICP) and right atrial pressure (RAP) of a non-breathing animal after a 35% blood loss. For sequential 5 minute time periods a vacuum is generated in the thorax by lowering airway pressures with an intrathoracic pressure regulator (ITPR) beginning with atmospheric pressure, then −5 mmHg, back to 0 mmHg, then −10 mmHg, and back to atmospheric or 0 mmHg. The top tracing where the ETP is shown. Large spikes represent positive pressure ventilations, including a showing that when ETP is decreased with the use of the ITPR, right atrial and intracranial pressures follow while aortic pressure shifts to the opposite direction leading to an increase of coronary and cerebral perfusion pressures. With each positive pressure ventilation, ICP is also transiently increased. These data show the relationship between increase and decreases in intrathoracic pressure and changes in intracranial pressure.

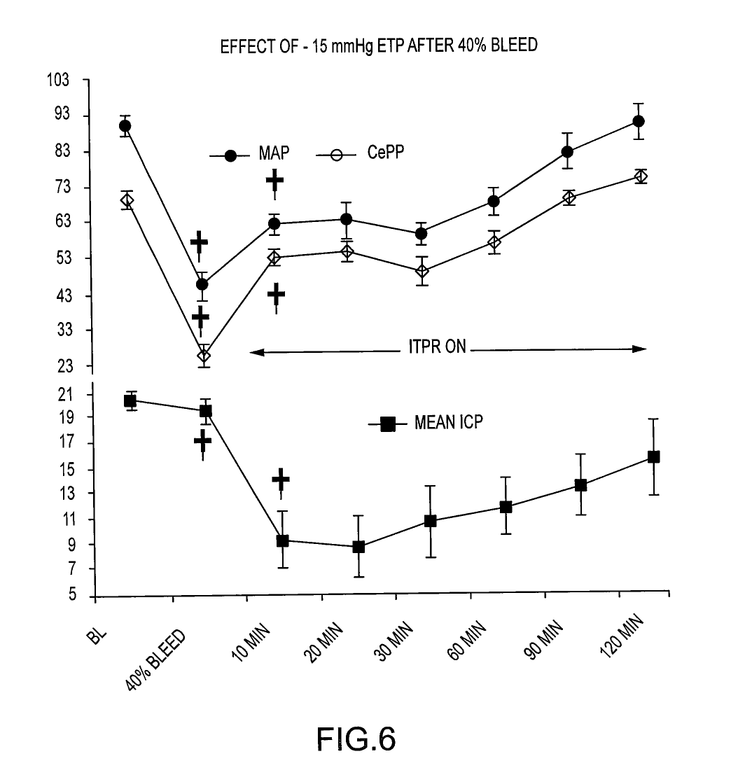

FIG. 6 illustrates that following controlled 40% bleed in a pig model of shock, an ITPR set at −15 mmHg significantly improves aortic pressure and decreases intracranial pressure, resulting in a significant increase of cerebral perfusion pressure for 2 hours: t means significant difference between the measured parameter at the two different time points (post bleed and 10 minutes) with a p<0.05. CePP is the cerebral perfusion pressure. ETP is the endotracheal or intrathoracic pressure. MAP is mean arterial pressure. ICP is intracranial pressure.

### EXAMPLES

The following examples are provided to demonstrate intrathoracic pressure regulation of intracranial pressure management in normovolemic and hypovolemic subjects. However, the invention is not limited by the described examples.

### Example 1

Both ITP and ICP can increase to a level that is dangerous during CPR when the chest is compressed but also not allow to expand laterally. The following experiment demonstrates a new observation related to the relationship between increases in ITP and the resultant increase in ICP. It further demonstrates the need for controlling and monitoring the force of compression applied during CPR so that cerebral perfusion pressures (calculated as the difference between the arterial pressure in the brain and the intracranial pressure) are maximized. These new concepts are shown by banding the chest with a circumference band. Banding the thorax increases intracranial pressure and central venous pressure leading to lower cerebral perfusion pressure and carotid flow independently from compression depth. This was demonstrated in 7 pigs in untreated ventricular fibrillation (VF) for 6 minutes followed by treatment with an automated compression device with a compression rate of 100/min, a depth of 25% of the anterior-posterior diameter, and 8 ventilations per minute (standard CPR) for 2 minutes. Thoracic banding was then added in the form of a 1.5 inch wide, leather belt, surrounding the mid thoracic cavity without impeding complete chest wall decompression. CPR was continued for 2 minutes with the banding and then stopped. Pressures within the trachea, thoracic aortic, intracranial (ICP), right atrial (central venous) pressures and common carotid blood flow were monitored and recorded continuously. Coronary perfusion pressure (CPP) was calculated as the diastolic aortic (Ao)−right atrial (RA) pressure. Cerebral perfusion pressure (CerPP) was calculated as CerPP=MAP−ICP. The mean tracheal pressure between positive pressure breaths, increased significantly from 4.8±1.2 with CPR alone to 6.2±1.7 with CPR+thoracic banding. The experiment showed that with this increase in intratracheal pressures, a surrogate for intrathoracic pressure, systolic aortic pressure with CPR alone versus CPR+thoracic banding was 52.3±5.6 versus 46.1±7.5 mmHg (p<0.05). Intracranial pressure with the above interventions was 23.7±1.6 with CPR alone versus 26.3±1.5 (p<0.05) with CPR+banding. The same response was observed with right atrial pressure. Cerebral perfusion and systemic perfusion pressures and were 14.9±4.8 and 16.6±5 with CPR alone versus 8.1±5 and 12.5±2.6 mmHg p<0.05 with CPR and thoracic banding, respectively. Common carotid blood flow in ml/min was: 80.5±18.5 versus 48.5±14.1 (p<0.05). These studies showed that with thoracic banding+CPR intrathoracic pressures were higher and this leads to significant decrease in systolic aortic pressure (as higher intrathoracic pressures inhibit venous blood flow back to the right heart) and an increase in ICP and RA pressure. These changes lead to a significant decrease in cerebral and systemic perfusion pressures and common carotid artery blood flow. Thoracic banding alters the chest wall mechanics in a way that adversely affects cerebral perfusion.

### Example 2

A more detailed description of Example 2 may be found in: Critical Care Medicine. 34(12) Suppl:S495-S500, December 2006. Yannopoulos, Demetris M D; McKnite, Scott H. B S; Metzger, Anja PhD; Lurie, Keith G. M D, incorporated herein by this reference. This example shows the newly discovered relationship between intracranial pressure and intrathoracic pressure. In this example, the pigs are not in cardiac arrest, as they were in Example 1.

Four different protocols were used to test the hypothesis that the two different methods (namely the inspiratory impedance threshold device or ITD and the intrathoracic pressure regulator or ITPR) to lower intrathoracic pressure would result in a decrease in intracranial pressure.

Protocol I: Spontaneously breathing sedated pigs with normal physiology were used to evaluate the effects of different negative intrathoracic pressures on intracranial pressures and cerebral perfusion pressure. For this protocol two difference inspiratory impedance threshold devices (ITD) (Advanced Circulatory Systems, Eden Prairie, Minn.) were used. One had a cracking pressures of −10 mmHg and the other −15 mmHg.

Protocol II: Following cardiac arrest and successful resuscitation, spontaneously breathing, sedated, pigs were used to evaluate the effect of negative intrathoracic pressure on post-arrest elevated intracranial pressure. Two different ITD cracking pressures of −10 and −15 mmHg were used, each in a random order.

Protocol III: The acute effects of an intrathoracic pressure regulator (ITPR) (Advanced Circulatory Systems, Eden Prairie, Minn.) on intracranial pressure and other physiological variables were evaluated in combination with a positive pressure mechanical ventilator in apneic hypovolemic hypotensive pigs.

Protocol IV: To determine if the effect of decreasing intracranial pressure by lowering intrathoracic pressure with an ITPR are sustained over time, intracranial and aortic pressures were measured in anesthetized and apneic pigs during a two hour application of the ITPR.

### Protocols:

### Protocol I.

Following a reduction in the dose of propofol anesthesia to allow for spontaneous ventilation to return to a stable state wherein pO₂ and pCO₂ values were normal, baseline ICP and central aortic pressure measurements were obtained. Next, an ITD was attached to the endotracheal tube for 15 minutes. A total of 6 animals were randomly assigned to an ITD with a cracking pressure of −10 mmHg and another 6 animals to an ITD with a cracking pressure of −15 mmHg.

### Protocol II.

After completion of the surgical preparation, ventricular fibrillation was induced by delivering a 50 Hz, 7.5 V AC electrical current via a temporary pacing wire positioned in the right ventricle. Following 4 minutes of untreated ventricular fibrillation animals (n=6) were successfully defibrillated with 1-3 biphasic electrical shocks of 150 Joules. After return to spontaneous of circulation and spontaneous respiration, ICP and aortic pressure were measured. Two ITDs were then attached in sequential order to the endotracheal tube, each for a 5 minute interval. The ITDs used had a cracking pressure of either −10 or −15 mmHg and were randomly assigned through a computer generated randomization list. After removal of the second ITD, the study was terminated with sacrifice of the animals.

### Protocol III.

Once the animals (n=12) were anesthetized, mechanically ventilated and in a steady state with normal pO₂ and ETCO₂ values for 5 minutes an ITPR was introduced between the ventilator tubing and the ET tube. The ITPR was used to regulate endotracheal pressures every 5 minutes in the following sequence: 0, −5, 0, −10, 0 mmHg. At the end, half of the animals were bled 35% of their blood volume and half were bled 50% at a rate of 60 ml/min over a period of 10-20 minutes. The ITPR was again applied in the same manner as before the hemorrhage and once previous sequence was repeated the animals were sacrificed.

### Protocol IV.

Animals (n=6) were anesthetized, mechanically ventilated, and bled 40% of their blood volume (as in Protocol II). Once the arterial blood gases demonstrated normal pO₂ and pCO₂ values for >5 minutes the ITPR was applied for 2 hours with positive pressure mechanical ventilation performed at a rate of 10 breaths/min with a tidal volume of 10 ml/kg and an FiO₂ of 0.5. The ITPR was used to maintain a negative endotracheal pressure of −15 mmHg and ICP was continuously recorded. At the end of the protocol animals were sacrificed.

### Results:

### Protocol I

Negative intrathoracic pressure generation during spontaneous breathing in 12 normovolemic pigs resulted in an instantaneous decrease in intracranial pressure. The negative intrathoracic pressure generated by the inspiratory effort of the animals resulted in a dose related decrease of the ICP during the inspiratory phase (Table 1). During spontaneous breathing through the ITD with the two different cracking pressures there was a dose related increase in mean arterial pressure and a dose related decrease in right atrial pressure as shown in the Table 1. The combination of an increase in mean arterial pressure and simultaneous decrease in right atrial and ICP resulted in a significant dose dependant increase of coronary and cerebral perfusion pressures.

TABLE 1

| | | MAP | ICP | CerPP | RA mean | CPP | Max neg ETP |
|---|---|---|---|---|---|---|---|
| −10 mmHg | Baseline | 86 ± 2.4 | 18.5 ± 1.7 | 67.9 ± 4.6 | −0.8 ± 1.5 | 90.4 ± 2.8 | −3.2 ± 0.5 |
| | Intervention | 96.7 ± 3.6 | 15.2 ± 0.8 | 81.5 ± 3.2 | −2 ± 2 | 100.7 ± 3.3 | −9 ± 1.3 |
| −15 mmHg | Baseline | 93.2 ± 4.9 | 17.7 ± 1.5 | 75.5 ± 5 | 0.5 ± 1.1 | 70.4 ± 5.6 | −4 ± 0.5 |
| | Intervention | 97.8 ± 4.1 | 13.5 ± 1.4 | 84.3 ± 4 | −4 ± 1.5 | 78.2 ± 4.4 | −18.2 ± 1.3 |

Table 1. Major hemodynamic parameters in normovolemic spontaneously breathing pigs breathing through an ITD of either −10 or −15 mmHg for 15 minutes.
* means significant difference between intervention (ITD on) and baseline (no ITD) with p < 0.05.
ETP: Endotracheal pressure;
AoP: aortic pressure;
ICP: intracranial pressure;
RA mean: mean right atrial pressure;
CerPP: Cerebral perfusion pressure;
CPP: coronary perfusion pressure;
Max neg ETP: maximum negative endotracheal pressure during inspiration.

### Protocol II

In protocol II, ICP after cardiac arrest in 6 pigs increased from 16.5±2 mmHg pre-arrest to 28.3±4.5 mmHg (p<0.001) immediately after restoration of spontaneous circulation. Cerebral perfusion pressure decreased from 78±11 mmHg before cardiac arrest to 41.7±7 mmHg follow return of spontaneous circulation after cardiac arrest (p<0.001). Post resuscitation, ICP with an ITD of −10 mmHg was decreased to 23±3 mmHg (p=0.05) and with −15 mmHg was decreased to 19±2.7 mmHg (p=0.03). Cerebral perfusion pressure increased to 51±6.7 (p<0.01) and 60±7.8 (p<0.001) mmHg with −10 and −15 mmHg ITD respectively compared to post resuscitation baseline.

### Protocol III

In protocol III use of ITPR to set airway pressure of apneic animals at −5, −10 mmHg resulted in a dose related decrease in ICP both during normovolemia and during hypovolemia. The effect is immediate and reproducible as shown in FIG. 5 from a real time tracing after a 35% bleed. The effects were significantly more pronounced during hypovolemia with the greater effect observed with 50% bleeding and −10 mmHg (Table 2). The opposite was true was well; positive pressure ventilation resulted in an increase in ICP. Application of negative airway pressure between PPVs resulted in a significant increase in mean arterial pressure and cerebral perfusion pressure with a significant volume dependant effect. Use of the ITPR set to −10 mmHg during 50% hypovolemia had the largest effect both on decreasing ICP and on increasing mean arterial pressure and cerebral perfusion pressure.

TABLE 2
Table 2. Major hemodynamic parameters during normovolemia
and after 35 and 50% blood loss with three different
endotracheal pressures of 0, −5 and −10 mmHg.

| | Hemodynamic | | | |
|---|---|---|---|---|
| % Blood | parameters | ETP (mmHg) | | |
| Loss | (mmHg) | 0 | −5 | −10 |
| 0% | MAP | 90 ± 4.5 | 101 ± 7.3* | 109 ± 6.1* |
| | RAP | 1.8 ± 0.4 | −0.8 ± 0.6* | −4.8 ± 0.4* |
| | CCP | 80 ± 3.9 | 94 ± 6.4* | 103 ± 5.3*† |
| | CerPP | 74 ± 4.8 | 88 ± 7.4* | 96 ± 6* |
| | ICP | 15.6 ± 0.6 | 13.5 ± 0.7 | 12.5 ± 0.6* |
| 35% | MAP | 46.5 ± 5 | 51 ± 4.7 | 55 ± 6.4* |
| | RAP | −0.1 ± 0.2 | −4.8 ± 0.5 | −8.2 ± 0.3*† |
| | CPP | 31 ± 3.4 | 45 ± 5.7 | 56 ± 5*† |
| | CerPP | 33 ± 4.2 | 41 ± 2.8 | 47 ± 5*† |
| | ICP | 14.1 ± 3.9 | 10.3 ± 2 | 7.6 ± 3*† |
| 50% | MAP | 29 ± 4.3 | 40 ± 5.9 | 47 ± 7.3*† |
| | RAP | −2 ± 0.9 | −5.7 ± 0.6 | −9.3 ± 0.3*† |
| | CPP | 26 ± 5 | 39 ± 3.7 | 48 ± 3.8*† |
| | CerPP | 18 ± 4.4 | 34 ± 5.8 | 45 ± 7.2*† |
| | ICP | 10.7 ± 1.3 | 6.2 ± 1.4 | 2.7 ± 1*† |

*means significant difference compared to ETP of 0 mmHg with p < 0.05 and
†means significant difference compared to ETP of −5 mmHg with p < 0.05.

The change of intracranial pressure seen with the use of ITPR is linearly correlated to circulating blood volume and endotracheal pressure (ETP) (surrogate of intrathoracic pressure) with the following equation: ΔICP=[1.22−0.84×(1-% blood loss/100)]×ETP.

### Protocol IV

The effect of continuous negative airway pressure on ICP was evaluated for up to 2 hours in Protocol IV. After the 40% bleed there was a small decrease in mean ICP from 20±2 to 19±1.5 mmHg and there was a significant decrease of mean arterial pressure from 90±2 to 45±5 mmHg. With the application of the ITPR (−15 mmHg) there was an initial significant decrease of ICP from 19±1.5 to 9±2 mmHg (p<0.01) and it was sustained below post bleed baseline for the whole duration of the application of −15 mmHg of ETP. There was significant increase of mean arterial pressure and cerebral perfusion pressure immediately with the application of −15 mmHg.

Having described the new scientific observations that form the basis for novelty, it will be recognized by those of skill in the art that various modifications, alternative constructions, and equivalents may be used without departing from the spirit of the invention. Additionally, a number of well known processes and elements have not been described in order to avoid unnecessarily obscuring the present invention. Accordingly, the above description should not be taken as limiting the scope of the invention.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a method” includes a plurality of such methods and equivalents thereof known to those skilled in the art, and so forth.

Also, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, acts, or groups.

What is claimed is:

1. A device for controlling rise in intracranial pressure (ICP) during cardiopulmonary resuscitation (CPR), the device comprising:
an intrathoracic pressure (ITP) sensor; and
a control system configured to adjust or alert for the need to adjust the amplitude and duration of CPR compressions to prevent a rise in ITP during a compression phase of CPR above a threshold amount, thereby controlling the rise in ICP during CPR.

2. The device of claim 1, wherein the threshold ITP amount is in the range from about 25 to about 50 mmHg, as measured in the thorax, including the trachea.

3. The device of claim 1, further comprising a mechanism for increasing cardiopulmonary and cerebral circulation through CPR compressions, wherein the mechanism is configured to allow for decompression after each compression.

4. The device of claim 3, wherein said mechanism is selected from the group consisting of a thoracic vest, a thoracic strap, a constant compression piston, an interposed abdominal compression (IAC) device, an active compression-decompression (ACD) device, manual compressions, an impedance threshold device (ITD), a limb compression device, and a combination IAC-ACD device.

5. The device of claim 1, wherein the ITP sensor is configured to sense ITP either directly or indirectly.

6. The device of claim 1, wherein the ITP sensor is integrated into an inspiratory impedance threshold (ITD) device, and the device further comprises said ITD device.

7. The device of claim 1, wherein the ITP sensor is configured to indirectly sense ITP, and comprises a bioimpedance sensor.

8. The device of claim 7, wherein the bioimpedance sensor is configured to detect the impedance in the neck or spine.

9. The device of claim 1, wherein the ITP sensor is configured to indirectly sense ITP, and comprises a blood pressure detector.

10. The device of claim 1, wherein the ITP sensor is configured to indirectly sense ITP, and comprises a pressure sensor for placement in the nose or on the neck.

11. The device of claim 1, further comprising a cerebral perfusion pressure sensor that is configured to sense cerebral perfusion pressures indirectly.

12. The device of claim 1, wherein the control system is configured to adjust or alert to adjust the amplitude and duration of CPR compressions to prevent a rise in ITP above the threshold amount during the compression phase.

13. The device of claim 1, wherein the control system is configured to adjust or alert to adjust the amplitude and duration of CPR compressions to prevent a rise in ITP above the threshold amount.

14. A device for controlling rise in intracranial pressure (ICP) during cardiopulmonary resuscitation (CPR), the device comprising:
a mechanism for increasing cardiopulmonary and cerebral circulation through CPR compressions, wherein the mechanism is configured to allow for decompression after each compression;
an intracranial pressure (ICP) sensor; and
a control system configured to adjust or alert to adjust the amplitude and duration of CPR compressions to prevent a rise in ICP above a threshold amount.

15. The device of claim 14, wherein said mechanism for increasing cardiopulmonary circulation through CPR compressions is selected from the group consisting of a thoracic vest, a thoracic strap, a constant compression piston, an interposed abdominal compression (IAC) device, an active compression-decompression (ACD) device, an impedance threshold device, manual compressions, limb compression device, and a combination IAC-ACD device.

16. The device of claim 14, wherein the ICP sensor is configured to sense ICP either directly or indirectly.

17. The device of claim 14, wherein the ICP sensor is configured to indirectly sense ICP or the change in ICP, and comprises a bioimpedance sensor.

18. The device of claim 17, wherein the bioimpedance sensor is configured to detect the impedance in the neck or spine.

19. The device of claim 14, wherein the ICP sensor is configured to indirectly sense ICP, and comprises a pressure monitor for placement in the nose or on the neck.

20. The device of claim 14, wherein the control system is configured to adjust or alert to adjust the amplitude and duration of CPR compressions to prevent an excessive rise in ICP during the chest compression phase.

21. The device of claim 14, wherein the control system is configured to adjust or alert to adjust the amplitude and duration of CPR compressions to prevent a decrease in cerebral perfusion pressures when the compression forces become too excessive.

22. A method for controlling the rise in intracranial pressure (ICP) during cardiopulmonary resuscitation (CPR), the method comprising:
performing CPR to a subject in need thereof;
directly or indirectly assessing ICP of the subject; and
adjusting the degree of CPR compressions if the subject's ICP is determined to be above a threshold amount.

23. The method of claim 22, wherein the subject's ICP is assessed via a device comprising an ICP sensor and/or an intrathoracic pressure (ITP) sensor; and a control system configured to adjust or alert to the need to adjust the degree of CPR compressions if the subject's ICP is determined to be above the threshold amount.

24. The method of claim 23, wherein CPR is performed via an automated compression device, and the control system is configured to automatically adjust the amplitude and duration of CPR compressions based at least in part on readings from a direct or indirect ICP and/or ITP sensor.

25. The method of claim 24, wherein the CPR is performed manually by a CPR administrator, and the control system visually and/or audibly alerts the CPR administrator to adjust the amplitude and duration of CPR compressions so as to prevent a rise in ICP based at least in part on readings from the ICP and/or ITP sensor.
